(19) United States
(12) Patent Application Publication
(10) Pub. No.: US 2012/0034617 A1
(43) Pub. Date: Feb. 9, 2012

(54) ASSAYS FOR BACTERIAL DETECTION AND IDENTIFICATION

(76) Inventors: Ambrose Lin-Yau Cheung, Hanover, NH (US); George Lee Newcomb, Norwich, VT (US)

(21) Appl. No.: 13/258,259

(22) PCT Filed: Mar. 31, 2010
(86) PCT No.: PCT/US10/29314

§ 371 Date: Oct. 24, 2011

Related U.S. Application Data
(60) Provisional application No. 61/165,556, filed on Apr. 1, 2009.

(51) Int. Cl. G01N 33/569; G01N 33/577; C12Q 1/68

(52) U.S. Cl. 435/6.15; 435/7.32; 435/7.34; 435/7.33; 435/7.37

(57) ABSTRACT

The invention herein generally relates to kits and methods for detecting the presence of a bacterium in a subject, for example, methicillin resistant S. aureus. In certain embodiments, the invention provides a method of detecting presence of a bacterium in a sample from a subject, the method including: contacting a sample from a subject with a bacterium-specific lytic enzyme or lysostaphin capable of specific lysis of a first bacterium if present in the sample, thereby exposing an intracellular gene or gene product of the first bacterium; contacting the sample with a particle having a protein on a surface of the particle in a presence of an antibody in which an Fc portion specifically binds the protein and an F(ab)2 portion specifically binds the intracellular gene or gene product of the first bacterium, with the proviso that when the particle is a second bacterium, the second bacterium is different from the first bacterium; and detecting the presence or absence of the first bacterium by observing the sample for an agglutination reaction, wherein agglutination indicates the presence of the first bacterium in the sample.

### TECHNICAL FIELD

The invention herein generally relates to compositions and methods for detecting bacteria. More particularly, the invention relates to compositions, methods, and kits for detecting and monitoring the presence of various types of bacteria, for example, methicillin-resistant S. aureus.

### BACKGROUND

Methicillin-resistant Staphylococcus aureus (MRSA) infections are the most common cause of noscomial or hospital-acquired infections (Archer, Clin. Infect. Dis. 26:1179, 1998). Incidence of MRSA infections has substantially increased over the last five years in healthy individuals without any known risk factors due to worldwide emergence of distinct MRSA strains known collectively as community acquired methicillin-resistant S. aureus (Groom et al., JAMA 286:1201-1205, 2001). Resistance to a greater number of antibiotics has occurred in S. aureus isolates worldwide. Besides common resistance to methicillin and β-lactams in general, S. aureus has also become resistant to drugs of last resort, such as vancomycin, linezolid, and daptomytin (Gale et al., Int. J. Antimicrob. Agents 27:300-302, 2006).

Currently, the diagnosis of MRSA relies on culture, chromogenic agar (Becton, Dickinson and Company), bacteriophage (Microphage, Inc.) assay, or PCR diagnostic for the mecA gene (Becton, Dickinson and Company and Cepheid) that encodes PBP2A. These diagnostic assays either require expensive and sophisticated equipment not commonly found in an emergency room and/or a physician's office (PCR) or entail a long processing time (from five hours in the bacteriophage assay to overnight in culture and chromogenic agar).

There is an unmet need for improved methods, kits and related reagents, and compositions for rapid detection and diagnosis of MRSA and other harmful bacteria in an emergency room and/or a physician's office.

### SUMMARY

The present invention is based, in part, on the discovery of an easy diagnostic test that is rapid (about 10 min. to about 15 min.), relatively inexpensive (about $40 per test or less), and does not require expensive and sophisticated instruments for diagnosis of presence and/or identification of bacterium in a sample, such as MRSA. The test is based on visually (or via instrumentation) observing agglutination, i.e., clamping, in a sample. Agglutination indicates a presence of the bacterium of interest in the sample. Lack of agglutination indicates an absence of the bacterium of interest in the sample.

The test can involve the following components: 1) a bacterium-specific lytic enzyme; 2) a body fluid or tissue sample from infected or colonization sites of a subject; 3) a particle having a protein on a surface of the particle, such, as Protein A. Protein G, or Protein L; 4) a monoclonal or highly specific polyclonal antibody in which an Fc portion of the antibody specifically binds the protein on the surface of the particle, and an F(ab)₂ portion of the antibody specifically binds the intracellular gene or gene product of the bacterium. The agglutinin consists of the particle and the antibody cross-linked with the intracellular gene or gene product released from the bacterium of interest in the sample.

An aspect of the invention provides a method of detecting presence of a bacterium in a sample from a subject. The method includes: contacting a sample from a subject with a bacterium-specific lytic enzyme (e.g., from a phage or another source capable of specific lysis of a first bacterium if present in the sample, thereby exposing an intracellular gene or gene product of the first bacterium; contacting the sample with a particle having a protein on a surface of the particle in a presence of an antibody in which an Fc portion specifically binds the protein and an F(ab)₂ portion specifically binds the intracellular gene or gene product of the first bacterium, with, the proviso that when the particle is a second bacterium, the second bacterium is different from the first bacterium; and detecting the presence or absence of the first bacterium by observing the sample for an agglutination reaction, wherein agglutination indicates the presence of the first bacterium in the sample. Prior to contacting the sample with the enzyme, the method can further include obtaining the sample from the subject.

Another aspect of the invention provides a method of identifying a bacterium in a sample from a subject. The method includes: aliquoting a sample into at least two vessels; contacting the sample in each vessel with a different bacterium-specific lytic enzyme (e.g., from, a phage or from another source), thereby exposing an intracellular gene or gene product of a first bacterium, in the vessel if the first bacterium is lysed by the particular enzyme added to that vessel; contacting the sample in each vessel with a particle having a protein on a surface of the particle, with the proviso that when the particle is a second bacterium, the second bacterium is not lysed by the enzyme that was added to that vessel; contacting the sample in each vessel with a different antibody, wherein the antibody added to each vessel is correlated with the enzyme that was added to that vessel; observing each vessel for presence of an agglutination reaction, wherein agglutination indicates presence of the first bacterium in that vessel; and identifying the first bacterium by correlating the vessel in which agglutination was observed with the enzyme or antibody that was added to the vessel. Prior to aliquoting, the method can further include obtaining the sample from the subject.

Another aspect of the invention provides a method of detecting presence of a bacterium in a sample from a subject. The method includes: contacting a sample from a subject with a bacterium-specific lytic enzyme (e.g., from a phage or from another source) capable of specific lysis of a first bacterium if present in the sample, thereby exposing an intracellular gene or gene product of the bacterium; inactivating the enzyme; contacting the sample with a second bacterium that over-expresses a surface protein in a presence of an antibody in which an Fc portion specifically binds the protein and an F(ab)₂ portion specifically binds the intracellular gene or gene product of the first bacterium; detecting the presence or absence of the first bacterium by observing the sample for an agglutination reaction, wherein agglutination indicates the presence of the bacterium in the sample. Prior to contacting the sample with the bacterium-specific lytic enzyme, the method can further include obtaining the sample from die subject, in certain embodiments, the first bacterium is different from the second bacterium. In other embodiments, the first bacterium is the same as the second bacterium.

Another aspect of the invention provides a method of identifying a bacterium in a sample from a subject. The method includes: aliquoting a sample into at least two vessels; contacting the sample in each vessel with a different bacterium-specific lytic enzyme (e.g., from a phage or another source), thereby exposing an intracellular gene or gene product of a first bacterium in the vessel if the first bacterium is lysed by the particular enzyme added to that vessel; inactivating the enzyme in each vessel; contacting the sample in each vessel with a particle having a protein on a surface of the particle; contacting the sample in each vessel with a different antibody, wherein the antibody added to each vessel is correlated with the enzyme that was added to that vessel; observing each vessel for presence of an agglutination reaction, wherein agglutination indicates presence of the first bacterium in that vessel; and identifying the first bacterium by correlating the vessel in which agglutination was observed with the enzyme or antibody that was added to the vessel. The particle and the antibody can be contacted to the sample simultaneously. Alternatively, the particle and the antibody can be contacted to the sample sequentially. Prior to contacting the sample with the bacterium-specific lytic enzyme (e.g., from a phage or another source), the method can further include obtaining the sample from the subject. In certain embodiments, the first bacterium is different from the second bacterium. In other embodiments, the first bacterium is the same as the second bacterium.

The particle can be a bead, such as a latex bead, that has a protein, such as Protein A, Protein G, Protein L, bound to a surface of the bead. Alternatively, the particle can be a second bacterium that over-expresses the protein. The second bacterium can be a heat-killed bacterium that over-expresses the protein or a live bacterium that over-expresses the protein. If the bacterium is a live bacterium, it should be an innocuous bacterium, i.e., harmless or benign to a subject, such as Lactococcos or Streptococcus gordonii. The sample can be a human tissue or body fluid, such as sputum, blood, urine, saliva, mucous, puss, or lymph.

The antibody can be a monoclonal antibody (e.g., murine, rabbit or human or humanized murine form) or a collection of monoclonal antibodies specific for different epitopes of the same intracellular gene product. Alternatively, the antibody is a highly specific polyclonal antibody.

Methods of the invention can be used to detect or identify bacterium selected from the group consisting of: methicillin-resistant S. aureus (MRSA), Group A Streptococcus (GAS), vancomycin resistant Enterococcus (VRE), Pneumococcus, Group B Streptococcus (GBS), and E. Coli OH: 157, Colostrum Difficile, and drug-resistant tuberculosis. In embodiments for detecting MRSA, the bacterium-specific lytic enzyme can be an S. aureus-specific phage lysin or lysostaphin, the antibody can be specific for a protein coming from a SCCmec cassette, such as PBP2A, and agglutination indicates the presence of MRSA in the sample.

Another aspect of the invention provides a method of determining presence of MRSA in a sample from a subject. The method includes: contacting a sample from a subject with an S. aureus-specific lytic enzyme to lyse S. aureus in the sample if present, thereby exposing an intracellular gene or gene product of the S. aureus; and detecting the presence of the intracellular gene or gene product by an immunoassay. The immunoassay can include a monoclonal antibody (e.g., murine, rabbit or human) or a collection of monoclonal antibodies specific for different epitopes of the same intracellular gene product. Alternatively, the immunoassay can include a polyclonal antibody.

The gene product can be a protein coming from an SCCmec cassette, such as PBP2A. The immunoassay can include agglutination of protein A or protein G in the immunoassay upon binding of the antibody to the gene or gene product if the S. aureus is present in the sample.

Another aspect of the invention provides a method of detecting presence of a bacterium in a sample from, a subject. The method includes: contacting a sample from a subject with a particle having a protein on a surface of the particle in a presence of an antibody in which an Fc portion specifically binds the protein on the surface of the particle and an F(ab)₂ portion specifically binds a cell surface protein or a secreted protein of a first bacterium; and detecting the presence or absence of the first bacterium by observing the sample for an agglutination reaction, wherein agglutination indicates the presence of the first bacterium in the sample. The particle and the antibody can be contacted to the sample simultaneously. Alternatively, the particle and the antibody can be contacted to the sample sequentially. Prior to contacting the sample with the particle and/or antibody, the method can further include obtaining the sample from the subject. The bacterium can be Clostridium Difficile, and E. Coli OH: 157.

Another aspect of the invention provides a method of determining presence of MRSA in a sample from a subject. The method includes: aliquoting a sample from a subject into a first aliquot and a second aliquot; contacting the first aliquot with an S. aureus-specific lytic enzyme to lyse S. aureus in the sample if present, thereby exposing an intracellular gene or gene product of the S. aureus, and detecting the presence of the intracellular gene or gene product by an immunoassay; contacting the second aliquot with an anti-coagulase antibody; and observing the first and second aliquots for presence of agglutination; wherein agglutination in both the first and second aliquots indicates presence of MRSA.

Another aspect of the invention provides a kit for detecting MRSA. The kit includes: S. aureus-specific lytic enzyme (e.g., from a phage or another source); at least one particle having a protein on a surface of the particle; and at least one antibody in which a Fc portion specifically binds the protein and a F(ab)₂ portion specifically binds an intracellular gene or gene product of S. aureus.

Another aspect of the invention provides a kit for detecting a bacterium. The kit includes: at least one bacterium-specific lytic enzyme (e.g., from a phage or another source); at least one particle having a protein on a surface of the particle; and at least one antibody in which a Fc portion specifically binds the protein and a F(ab)₂ portion specifically binds an intracellular gene or gene product of a bacterium lysed by the enzyme. The at least one bacterium-specific lytic enzyme can be a plurality of different bacterium-specific lytic enzymes, in which each enzyme specifically lyses a different bacterium. The at least one antibody can be a plurality of different antibodies, each of the antibodies having a specificity for a particular gene or gene product unique to a particular bacterium.

### BRIEF DESCRIPTION OF THE DRAWINGS

FIG. 1 is a diagram schematically depicting release of intracellular genes or gene products from a target bacteria using a bacterium-specific lytic enzyme (e.g., from a phage or from other bacteria).

FIG. 2 is a diagram schematically depicting generation of an agglutination platform.

FIG. 3 is a diagram schematically depicting agglutination consisting of a particle and an antibody cross-linked by an intracellular gene or gene product of a specific bacterium.

FIG. 4 depicts exemplary expression and localization of protein A in L. lactis.

FIG. 5 shows exemplary binding of a fixed number of protein A-expressing L. lactis cells to FITC-conjugated IgG from different mammalian species.

FIG. 6 depicts purification of PBP2a.

FIG. 7 depicts agglutination reactions of anti-OVA antibody attached to protein A-expressing L. lactis upon addition of OVA antigen.

### DETAILED DESCRIPTION

The invention herein generally relates to novel and improved methods, kits and reagents, and compositions for detecting and monitoring the presence of various bacteria in a subject, for example, methicillin resistant S. aureus (MRSA). In certain embodiments, methods of the invention involve contacting a sample from a subject with a bacterium-specific lytic enzyme (from a phage or another source) capable of specific lysis of a particular bacterium if present in the sample, thereby exposing an intracellular gene or gene product of the particular bacterium.

The sample can be a mammalian, e.g. human, tissue or body fluid. A tissue is a mass of connected cells and/or extracellular matrix material, e.g. skin tissue, nasal passage tissue, CNS tissue, neural tissue, eye tissue, liver tissue, placental tissue, mammary gland tissue, gastrointestinal tissue, musculoskeletal tissue, genitourinary tissue, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, phlegm, saliva, sweat, amniotic fluid, mammary fluid, and cerebrospinal fluid (CSF), such as lumbar or ventricular CSF. A sample also may be media containing cells or biological material.

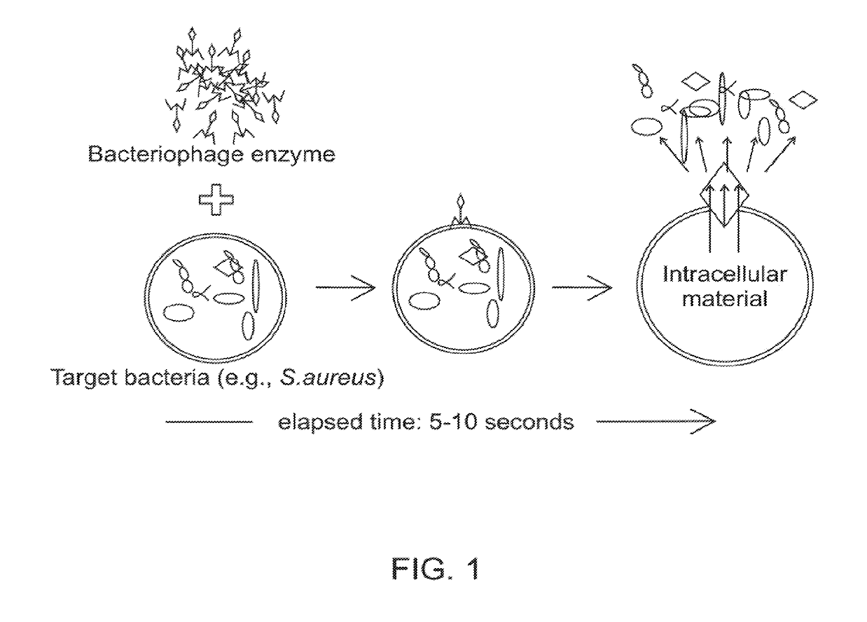

Lytic enzymes are highly evolved enzymes produced by a bacteriophage (phage) or bacteria (e.g. lysostaphin produced by Staphylococcus simulans) to digest the bacterial cell wall. In Gram-positive bacteria, small quantities of purified recombinant lysin added externally results in immediate lysis causing log-fold death of the target bacterium. Advantages of these lytic enzymes from phage or bacteria include specificity for a particular bacteria without lysing other bacteria present in a sample (Fishetti, Curr. Opi. Microbiol., 11:393-400, 2008) (Recsei, PNAS, 5:1127-1131, 1987). FIG. 1 is a diagram schematically showing a bacterium-specific lytic enzyme (from a phage or another bacterium) binding to a target bacterium, for example S. aureus, and disrupting the cell wall of the bacterium. Once the cell wall is breached, the inner membrane of the bacterium cannot hold the intracellular material and the bacterium bursts, releasing the intracellular material, including intracellular genes and typically gene products, of the bacterium into the sample. The entire process from binding to lysing occurs rapidly, for example, in about 10 seconds, in about 30 seconds, in about 1 minute, in about 2 minutes, in about 3 minutes, etc. Lysins from DNA-phage that infect Gram-positive bacteria are generally between 25 and 40 kDa in size except the PlyC for streptococci that is 114 kDa. This enzyme is unique because it is composed of two separate gene products, PlyCA and PlyCB (Fishetti, Curr. Opt. in Microbiol., 11:393-400, 2008). With some exceptions, the N-terminal domain contains the catalytic activity of the enzyme. This activity may be either an endo-b-N acetylglucosaminidase or Nacetylmuramidase (lysozymes), both of which act on the sugar moiety of the bacterial wall, an endopeptidase that acts on the peptide moiety, or an N-acetylmuramoyl-L alanine amidase (or amidase), which hydrolyzes the amide bond connecting the glycan strand and peptide moieties (Young, Microbiol. Rev., 56:430-481, 1992; and Loessner, Curr. Opi. Microbiol., 8:480-487, 2005). In some cases, particularly staphylococcal lysins, two and perhaps even three different catalytic domains may be linked to a single binding domain (Navarre et al., J. Biol. Chem., 274:15847-15856, 1999).

Studies of lysin-treated bacteria reveal that lysins exert their effects by forming holes in the cell wall through peptidoglycan digestion (Fishetti, Curr. Opi. Microbiol., 11:393-400, 2008). The high internal pressure of bacterial cells (roughly 3 to 5 atmospheres) is controlled by the highly cross-linked cell wall. Any disruption in the integrity of the wall will result in extrusion of the cytoplasmic membrane and ultimate hypotonic lysis (Fishetti, Curr. Opi. Microbiol., 11:393-400, 2008). In certain embodiments, a single enzyme molecule is used to cleave an adequate number of bonds to kill a target bacterium.

In general, lysins only kill the species (or subspecies) of bacteria from which they were produced (Fishetti, Curr. Opi. Microbiol., 11:393-400, 2008). For instance, enzymes produced from streptococcal phage kill certain streptococci, and enzymes produced by pneumococcal phage kill pneumococci (Nelson et al., Proc. Nat'l Acad. Sci. USA, 98:4107-4112, 2001; and Loeffler et al. Science, 294:2170-2172, 2001). Specifically, a lysin from a group C streptococcal phage (PlyC) will kill group C streptococci as well as groups A and E streptococci, the bovine pathogen S. uberis and the horse pathogen, S. equi, without effecting streptococci normally found in the oral cavity of humans and other Gram-positive bacteria (Fishetti, Curr Opi Microbiol, 11:393-400, 2008). Similar results are seen with a pneumococcal specific lysin (Fishetti, Curr. Opi. Microbiol., 11:393-400, 2008).

An important lysin with respect to infection control is a lysin directed to S. aureus. A staphylococcal enzyme and methods of producing the enzyme is described in Fishetti (Curr. Opi. Microbiol., 11:393-400, 2008) and Rashel et al. (J. Infect. Dis., 196:1237-1247, 2007). This lysin is easily produced recombinantly and has a significant lethal effect on MRSA both in vitro and in a mouse model (Rashel et al., J. Infect. Dis., 196:1237-1247, 2007).

Lysins that specifically lyse Group A Streptococcus (GAS), vancomycin resistant Enterococcus (VRE), Pneumococcus, Group B Streptococcus (GBS), and Bacillus anthracis are also shown in Fishetti (Curr. Opi. Microbiol., 11:393-400, 2008).

In the case of S. aureus, lysostaphin can also be an effective lytic enzyme, Lysostaphin is produced by Staphylococcus simulans. The proenzyme has a molecular weight of about 42 kDa. The mature enzyme is about 25-28 kDa and is a zinc metalloprotease that is capable of cleaving the glycyl-glycine bond of the pentaglycine crossbridge linking different strands of peptidoglycan (Recsei, PNAS, 5:1127-1131, 1987), resulting in an un-crosslinked cell wall, and hence leading to cell lysis. The effect is specific for S. aureus.

Upon lysis of the target bacterium, the intracellular genes or gene products are released into the sample. Included are intracellular genes and gene products that are specifically associated with the target bacterium, and unique to that bacterium, allowing for subsequent identification of the bacterium in the sample, as discussed further below. A gene product includes biochemical material, for example RNA or protein, resulting from expression of a gene.

All S. aureus isolates, both methicillin sensitive and resistant strains, carry three high molecular weight penicillin binding domains (PBP), PBP1, PBP2, and PBP3, to which most β-lactam antibiotics bind, and a low molecular weight PBP called PBP4 that binds poorly to most β-lactams. PBP1 and PBP2 are important enzymes involved in synthesis of bacterial cell wall; the β-lactam antibiotics generally kill bacteria interfering with the transpeptidase domain of penicillin binding proteins, that leads to a loss of cell-wall cross-linking integrity (Mallorqui-Fernandez et al., FEMS Microbiol. Lett. 235:1-8, 2004). PBP4, a single low molecular weight PBP, has been shown to have a low affinity for most β-lactams, and is unique among low-molecular weight PBPs found among prokaryotes in that it possesses transpeptidase and carboxypeptidase activities (Kozarich et al, J. Biol. Chem. 253:1272-1278, 1978).

Methicillin resistance is achieved by acquisition of another high molecular weight PBP, namely PBP2A encoded by mecA, situated in the chromosome in a genomic island designated staphylococcal cassette chromosome mec (SSCmec). Unlike innate penicillin binding proteins, PBP2A has a remarkably low affinity for all β-lactams (Matsuhashi et al., J. Bacterial 167:975, 1986).

Group A Streptococcus (GAS) is a bacterium often found in the throat and on the skin. People may carry GAS in the throat or on the skin and have no symptoms of illness. Most GAS infections are relatively mild illnesses such as strep throat, or impetigo. Occasionally these bacteria can cause severe and even life-threatening diseases.

Severe, sometimes life-threatening, GAS disease may occur when bacteria get into parts of the body where bacteria usually are not found, such as the blood, muscle, or the lungs. These infections are referred to as invasive GAS disease. Two of the most severe forms of invasive GAS disease are necrotizing fasciitis and streptococcal toxic shock syndrome. Necrotizing fasciitis is a rapidly progressive disease that, destroys muscles, fat, and skin tissue. Streptococcal toxic shock syndrome (STSS) results in a rapid drop in blood pressure and organs (e.g., kidney, liver, lungs) to fail STSS is not the same as the toxic shock syndrome due to the bacteria S. aureus that has been associated with tampon usage. While 10% to 15% of patients with invasive GAS disease die from their infection, approximately 25% of patients with necrotizing fasciitis and more than 35% with STSS die.

GAS produces many virulence factors that promote survival in humans, A two-component regulatory system, designated covRS (cov, control of virulence: csrRS), negatively controls expression of five proven or putative virulence factors (capsule, cysteine protease, streptokinase, streptolysin S, and streptodornase). Graham et al., PNAS, 99(21): 13855-13860, 2002. Additional genes and gene products of GAS are shown in Viraneve et al. (Infect. Immun., 71(4):2199-2207, 2003), Ferretti et al. (Proc. Natl. Acad. Sci. USA, 98:4658-4663, 2001), and Lloyd (J. Med. Microbiol., 56:1574-1575, 2007).

Group B Streptococcus (GBS) is a very common cause of sepsis (blood infection) and meningitis (infection of the fluid and lining around the brain) in newborns. GBS is also a frequent cause of newborn pneumonia. Putative adherence genes, designated as sspB1 and sspB2, encode proteins homologous to the broad family of adherence and aggregation proteins commonly found in Gram-positive bacteria (Suvorov et al. International Congress Series, 1289:227-230, 2006). The occurrence of sspB1 and sspB2 variants is correlated with invasive GBS strains (Suvorov et al. International Congress Series, 1289:227-230, 2006). Additional genes and gene products of GBS are shown in Kong et al. (J. Clinical Microbiology, 40(2):620-626, 2002) and Zhao et al. (Clin. Microbiol. Infect., 14(3):260-267, 2008).

Enteroccocci are bacteria that are normally present in the human intestines and in the female genital tract and are often found in the environment. These bacteria can sometimes cause infections. Vancomycin is an antibiotic that is often used to treat infections caused by Enterococci. In some instances, Enterococci have become resistant to this drug and thus are called vancomycin-resistant Enterococci (VRE). Most VRE infections occur in hospitals.

VRE can be conferred by one of two functionally similar operons, vanA or vanB, as shown in Arthur et al. (Trends Microbiol, 4:401-407, 1996). vanA and vanB operons are highly sophisticated resistance determinants, that suggests that they evolved in other species and were acquired by Enterococci. The difference in the guanine-cytosine (G-C) content of the genes of the vanB operon (roughly 50% G-C; Evers, Gene., 124:143-144, 1993) in comparison to typical Enterococcal genes (35% to 40% G-C; Murray, Clin. Microbiol. Rev., 3:46-65, 1990) is compelling evidence for this acquisition.

More than 95% of VRE recovered in the United States are E. faecium; virtually all are resistant to high, levels of ampicillin. Ampicillin resistance in E. faecium is attributable to the production of a low-affinity penicillin-binding protein, PBP5 (Fontana et al, J. Bacteriol., 155:1343-1350, 1983). Further genes and gene products associated with VRE are shown in Patino et al. (J. of Bacteriol., 184(23):6457-6464, 2002).

Pneumococcal disease caused by Streptococcus pneumoniae is a leading cause of serious illness in children and adults throughout the world. Pneumococcal invasion of the lungs results in community-acquired bacterial pneumonia. Pneumococcal invasion of the bloodstream results in bacteremia, and Pneumococcal invasion of the covering of the brain results in meningitis. Pneumococci may also cause otitis media (middle ear infection) and sinusitis. Currently there are more than 90 known Pneumococcal types, and the ten most common types account for approximately 62% of invasive disease worldwide.

Penicillin-resistant strains of Pneumococcus have been correlated with the pbp2x gene (Hakenbeck et al. Infect Immun., 69(4):2477-2486, 2001). Additional genes and gene products of Pneumococcus are shown in Orihuela et al. (Infection and Immunity, 72(10):5582-5596, 2004) and Suzuki et al. (J. Med. Microbiol, 55:709-714, 2006).

Bacillus anthracis is a gram-positive spore-forming bacterium that causes the disease anthrax. The anthrax toxin contains three components, including the protective antigen (PA), that binds to eukaryotic cell surface receptors and mediates the transport of toxins into the cell (Price et al., J. of Bacteriol., 181(8):2358-2362, 1999). The main toxic genes are pagA, lef and cya, and the genes related to capsule synthesis are capA, capB and capC. Additional genes and gene products of Bacillus anthracis are shown in Price et al. (J. of Bacteriol., 181(8):2358-2362, 1999) and Shard et al. (J. Bacteriol., 176(16):5188-5192, 1994).

Table 1 below provides phage-lytic enzymes that lyse particular bacteria, and intracellular genes and gene products of interest.

TABLE 1

| | Phage | Target Gene | |
|---|---|---|---|
| Pathogen | Enzyme | Product | References |
| MRSA | ClyS | PBP2A | Fishetti, Curr Opi Microbiol 11: 393-400, 2008 |
| | | | Rashel et al., J Infect Dis, 196: 1237-1247, |
| | | | 2007 |
| Group B Strep | PlyGBS | cspA or surface | Cheng et al., Antimicrob Agents Chemother, |
| | | polysaccharide | 49: 111-117, 2005 |
| | | | Harris et al., J. Clin Invest, 111: 61-70, 2003 |
| Group A Strep | PlyC | M protein in the | Fischetti, Trends in Mocrob, 13: 491-496, 2005 |
| | | constant region | Robbins et al., J. Bacteriol., 169: 5633-5640; |
| | | | 1987 |
| Pneumococcus | Cpl-1 | CpsA, CpsB, CpsC | Loeffler et al. Infect Immun, 71: 6199-6204, |
| | | and CpsD | 2003 |
| | | | Yu et al., J. Medical Microbiology, 57: 171- |
| | | | 178, 2008 |
| Vancomycin | PlyV12 | VanA or VanB | Yoong et al. J. Bacteriol., 186: 4808-4812, |
| Resistant | | | 2004 |
| Enterococcus | | | Joong-Sik et al. J. Clin Microbiology, 1785- |
| | | | 1786, 2004 |
| Bacillus | PlyG | protective antigen | Fishetti, Curr Opi Microbiol, 11: 393-400, 2008 |
| anthracis | | (PA), lethal factor | |
| | | (LF), and edema | |
| | | factor (EF) | |
| drug resistant | Che12 | Lipoarabhiomannan | Kumar et al., Tuberculosis, 88: 616-623, 2008 |
| tuberculosis | | determines TB or | Marttila et al. Antimicrobial Agents and |
| | | KatG = sensitive | Chemotherapy, 40: 2187-2189, 1996 |
| | | no KatG = resistant | |

After lysing the bacterium in the sample with the bacterium-specific lytic enzyme to expose the intracellular genes or gene products of the particular bacterium, the sample is contacted with a particle having a protein on a surface of the particle. In certain embodiments, the gene product of the particular bacterium is present on the surface of the cell or is secreted. In embodiments in which the gene product is present on the surface of the cell or is secreted, it is not necessary to contact, the sample with a bacterium-specific lytic enzyme. Instead, the sample can simply be contacted with a particle having a protein on a surface of the particle. Exemplary bacteria that contain cell surface proteins that would allow for identification of the bacteria without first lysing the bacteria include Escherichia coli and Clostridium difficile. A protein of interest of E. coli is Shiga-like toxin (Zhao et. al., Antimicrobial Agents and Chemotherapy, 1522-1528, 2002). A protein of interest of C. difficile is Exotoxin A and B (Sifferta et al. Microbes & Infection. 1159-1162, 1999).

The particle can be any type of particle that has a surface protein, such as Protein A, Protein G, or Protein L, or is capable of be coupled to a surface protein, such as Protein A, Protein G, or Protein L. Exemplary particles include beads that are capable of being coupled with the surface protein, such as latex beads, resin beads, magnetic beads, gold beads, polymer beads, or any type of bead known in the art. The bead has a protein, such as Protein A, Protein G, or Protein L, coupled to the surface of the bead. Methods for coupling proteins to the surface of beads are known in the art. See, e.g., Sambrook, et al. Molecular Cloning—A Laboratory Manual, Cold Spring Harbor Laboratory, Cold Spring Harbor, N.Y. (1985). The protein can be covalently coupled to the surface of the bead or non-covalently coupled, e.g., hydrogen bonding, ionic bonding, or Van der Waals bonding, to the surface of the bead.

In particular embodiments, the protein coupled to the bead is Protein A or Protein G. Protein A and Protein G bind to the Fc region of immunoglobulins, leaving the antigen binding Fab region unhindered. Beads with Protein A or Protein G coupled to the surface are commercially available from Invitrogen (Carlsbad, Calif.).

The particle can also be a live or heat killed bacterium that has been engineered with a recombinant plasma to over-express a surface protein, such as Protein A, Protein G, or Protein L. A heat-killed bacterium refers to a bacterium that has been killed by heating, yet structure and integrity of the proteins on the surface of the bacterium have been maintained, thus preserving the function of these proteins to bind other molecules, such as antibodies. An exemplary procedure for heat-killing a bacterium while still maintaining the structure and integrity of the surface proteins involves heating the bacterium at about 55° C. for one hour. The heat-killed bacterium can be any bacterium, in order to enhance a person's ability to visualize the agglutination reaction, the heat-killed bacterium can be stained with a dye after heat-killing. The dye can be any color dye that can be visualized be the human eye, for example green, blue, yellow, orange, red, etc.

The live bacterium should be an innocuous bacterium. An innocuous bacterium, or a harmless or benign bacterium, refers to a bacterium that will not adversely effect, harm, or injure a subject that comes in contact with or handles the bacterium. Exemplary innocuous bacterium include Lactococcus or Streptococcus gordonii, Lee et. al. (Microbes and infection, 11:20-28, 2009) discusses use of Lactococcus or Streptococcus gordonii as live antigen delivery vehicles.

In certain embodiments, the particle is Lactococcus that has been transfected with a vector containing a protein A gene from S. aureus. There are many benefits to using Lactococcus transfected with a vector containing a protein A gene from S. aureus as the vector for the agglutination reactions, such as: the protein A gene from S. aureus varies with respective to the number of binding sites (up to seven) for the F(c) portion of an IgG antibody; different strains of S. aureus express different (larger) protein A gene products; Lactococcus can be readily manipulated on a molecular genetic scale to accommodate protein A on its surface (high plasmid copy number (up to 15) yields more protein A expression, and choice of 38 different promoters optimizes promoter strength for best expression); protein A binds the F(c) portion of the antibody producing the correct orientation of the F(ab)₂ portion of the antibody for binding intracellular genes and gene products or cell surface gene products; multiple monoclonal antibodies bind to different sites on the target protein (e.g., PBP2a), dramatically increasing the agglutination; and the amount of protein A-expressing Lactococcus in solution that binds PBP2a specifically can be increased dramatically and cheaply to increase sensitivity. The cumulative effect of these factors is that the Lactococci can be engineered with increased binding ability for agglutination reaction diagnostics.

In certain embodiments, the live or heat killed bacterium should be a bacterium that is unaffected by the bacterium-specific lytic enzyme, i.e., is not lysed by the enzyme. Thus the live or heat-killed bacterium should be different from the bacterium that is to be detected by the methods of the invention. For example, if the sample is being tested for presence of MRSA, the live or heat-killed bacterium to be contacted to the sample can be any bacterium except MRSA, such as Lactococcus, Streptococcus gordonii, Group A Streptococcus, Enterococcus, Pneumococcus, Group B Streptococcus, or Bacillus anthracis.

in other embodiments, the live or heat-killed bacterium can be any bacterium, even a bacterium that is the same as the bacterium for which the presence in the sample is being investigated. For example, if the sample is being tested for presence of MRSA, the live or heat-killed bacterium to be contacted to the sample can be any bacterium, including methicillin-sensitive Streptococcus aureus or MRSA. In these embodiments, the sample is contacted with an agent that inactivates the bacterium-specific lytic enzyme, prior to the sample being contacted by the live or heat-killed bacterium. Thus the live or heat-killed bacterium is not effected, i.e. not lysed, by the bacterium-specific lytic enzyme because the enzyme has been inactivated. Inactivation of the bacterium-specific lytic enzyme can be accomplished by any method known in the art, such as adding a buffer to the sample that inactivates the enzyme or adding an enzyme inhibitor to the sample.

The live or heat killed bacterium are engineered to over-express a surface protein, such as Protein A, Protein G, or Protein L. Over-expression of a surface protein by the live or heat-killed bacterium is accomplished by methods known in the art. Exemplary vectors and methods for over-expressing a surface protein, in particular protein A and Protein G, in live or heat-killed bacterium are shown in Provvedi et al. (BMC Biotechnology, 5:3, 2005), Song et al. (Biotechnol. Lett., 2009), Zhao et al. (Biotechnology Advances 24:285-295, 2006), Nouailie et al. (Genet. Mol. Res., 2(1): 102-111, 2003), Myscofski et al. (Protein Expression and Purification 14:409-417, 1998), Oggioni et al. (Gene, 169:85-90, 1996), and Guimaraes et al. (Genetic Vaccines and Therapy, 7:4, 2009).

The sample is also contacted with an antibody in which an Fc portion of the antibody specifically binds the protein on the surface of the particle, and an F(ab)₂ portion of the antibody specifically binds the intracellular genes or gene products of the bacterium that has been lysed. The term “antibody” as referred to herein includes whole antibodies and any antigen binding fragment (i.e., “antigen-binding portion”) or single chains of these. A naturally occurring “antibody” is a glycoprotein including at least two heavy (H) chains and two light (L) chains inter-connected by disulfide bonds.

As used herein, an antibody that “binds genes or gene products of the bacterium that has been lysed” is intended to refer to an antibody that binds to genes or gene products of the bacterium that has been lysed with a K_{D} of 5×10⁻⁹ M or less, 2×10⁻⁹ M or less, or 1×10⁻¹⁰ M or less. For example, the antibody is monoclonal or polyclonal. The terms “monoclonal antibody” or “monoclonal antibody composition” as used herein refer to a preparation of antibody molecules of single molecular composition. A monoclonal antibody composition displays a single binding specificity and affinity for the genes or gene products of the bacterium that has been lysed or for a particular epitope of the genes or gene products of the bacterium that has been lysed. The antibody is an IgM, IgE, IgG such as IgG1 or IgG4. The monoclonal antibody can be sources from rabbit, human or murine origin or chimera such as humanized murine monoclonal antibodies. In our studies, rabbit and human antibodies are found more tightly to protein A bound to L. Lactococcus.

Also useful is an antibody that is a recombinant antibody. The term “recombinant human antibody”, as used herein, includes all antibodies that are prepared, expressed, created or isolated by recombinant means, such as antibodies isolated from an animal (e.g., a mouse). Mammalian host cells for expressing the recombinant antibodies used, in the methods herein include Chinese Hamster Ovary (CHO cells) including dhfr-CHO cells, described in Urlaub and Chasin, Proc. Natl. Acad. Sci. USA 77:4216-4220, 1980 used with a DH FR selectable marker, e.g., as described in R. J. Kaufman and P. A. Sharp, 1982 Mol. Biol. 159:601-621, NSO myeloma cells, COS cells and SP2 cells. Another expression system is the GS gene expression system shown in WO 87/04462, WO 89/01036 and BP 338,841. To produce antibodies, expression vectors encoding antibody genes are introduced into mammalian host cells or yeast, and the host-cells are cultured for a period of time sufficient to allow for expression of the antibody in the host cells or secretion of the antibody into the culture medium in which the host cells are grown. Antibodies can be recovered from the culture medium using standard protein purification methods.

Standard assays to evaluate the binding ability of the antibodies toward the target of various species are known in the art, including for example, ELISAs, western blots and RIAs. The binding kinetics (e.g., binding affinity) of the antibodies also can be assessed by standard assays known in the art, such as by Biacore analysis.

General methodologies for antibody production, including criteria to be considered when choosing an animal for the production of antisera, are described in Harlow et al. (Antibodies, Cold Spring Harbor Laboratory., pp. 93-117, 1988). For example, an animal of suitable size such as goats, dogs, sheep, mice, rabbit or camels are immunized by administration of an amount of immunogen, such as the intact, protein or a portion thereof containing an epitope from a genes or gene products of the bacterium that has been lysed, effective to produce an immune response. An exemplary protocol is as follows. The animal is subcutaneously injected in the back with 100 micrograms to 100 milligrams of antigen, dependent on the size of the animal, followed three weeks later with an intraperitoneal injection of 1.00 micrograms to 100 milligrams of immunogen with adjuvant dependent on the size of the animal, for example Freund's complete adjuvant. Additional intraperitoneal injections every two weeks with adjuvant, for example Freund's incomplete adjuvant, are administered until a suitable titer of antibody in the animal's blood is achieved. Exemplary titers include a titer of at least about 1:10,000 (30 or a titer of 1:100,000 or mote, i.e., the dilution having a detectable activity. The antibodies are purified, for example, by affinity purification on columns containing hepatic cells.

The technique of in vitro immunization of human lymphocytes is used to generate monoclonal antibodies. Techniques for in vitro immunization of human lymphocytes are well known to those skilled in the art See, e.g., Inai, et al., Histochemistry, 99(5):335 362, May 1993; Mulder, et al., Hum. Immunol, 36(3):186 192, 1993; Harada, et al, J. Oral. Pathol. Med., 22(4): 145 152, 1993; Stauber, et al., J. Immunol. Methods, 161(2): 157 168, 1993; and Venkateswaran, et al. Hybridoma, 11(6) 729 739, 1992. These techniques can be used to produce antigen-reactive monoclonal antibodies, including antigen-specific IgG, and IgM monoclonal antibodies, in the case of human monoclonal antibodies, they can be produced from yeast cells carrying a library of various antigenic determinants. Any antibody or fragment thereof having affinity and specific for the genes or gene products of the bacterium that has been lysed is within the scope of the invention provided herein.

After contacting the sample with the particle and the antibody, the sample is visually observed for an agglutination reaction. The agglutination indicates the presence of the bacterium of interest in the sample. Agglutination refers to the clumping of particles. The agglutinin will consist of the particle and the antibody cross-linked with the intracellular gene or gene product released from the bacterium in the sample.

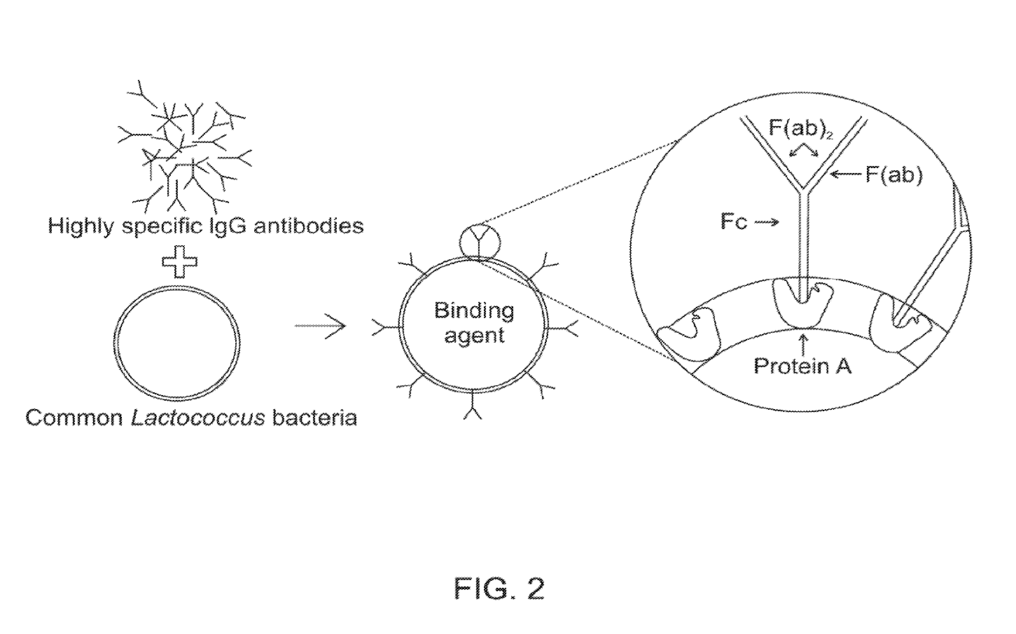

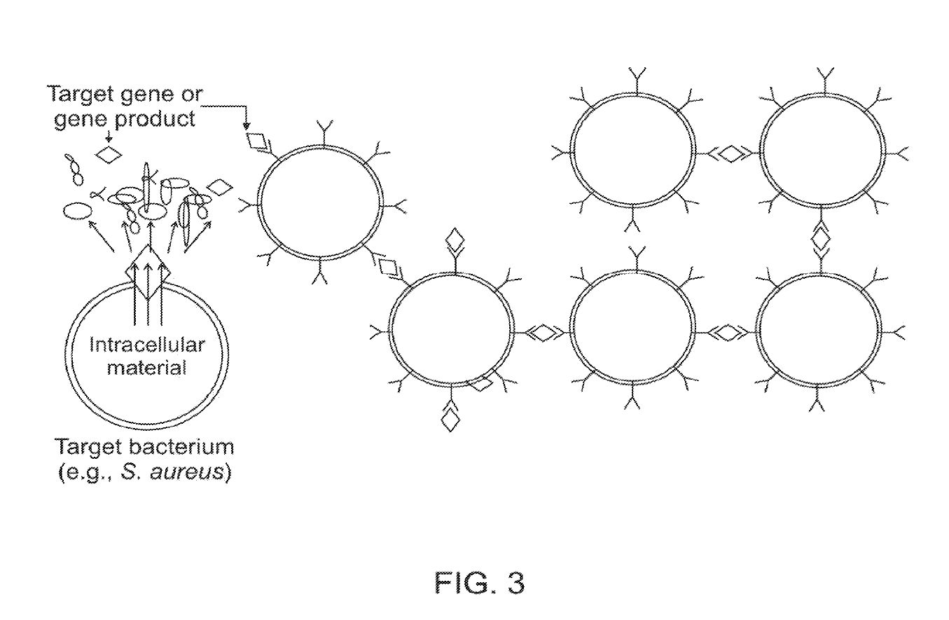

FIGS. 2-3 depict aspects of the agglutination reaction. FIG. 2 shows the Fc portion of the antibody interacting with the protein, for example Protein A or Protein G, on the surface of the particle. It is known that Protein A and Protein G have a high affinity for the Fc portion of antibodies, for example IgG. Thus the particles having the surface protein, such as Protein A or Protein G, bind the Fc portion of the antibody in the sample. Because the Fc portion of the antibody interacts with the surface protein, the antigen-binding F(ab)₂ portion of the antibody is oriented outward, thus displaying the antigen-binding F(ab)₂ portion of the antibody to interact with the intracellular genes and gene products of the lysed bacterium (FIG. 2).

FIG. 3 shows the intracellular genes and/or gene products interacting with, the antigen-binding F(ab)₂ portion of the antibody, in which the Fc portion of the antibody is interacting with the protein coupled to the surface of the particle, thus forming the agglutinin. Cross-linking occurs because multiple antibodies can bind the same intracellular gene or gene product. (FIG. 3). The gene or gene product forms the cross-link between the antibody bound particles. This cross-linking results in agglutination, i.e. clumping, which will rapidly fall out of the aqueous solution, and form a visible precipitate indicative of the presence of the target bacterium (FIG. 3).

Another aspect of the invention provides a method for identifying an unknown bacterium in a sample from a subject. In this embodiment, the sample is aliquoted into multiple vessels. The vessel can be any type of vessel that is capable of holding a sample. An exemplary vessel is a microtiter plate. A different bacterium-specific phage lysing enzyme is then added to each sample in each vessel. Because each enzyme only lyses a particular bacterium, the bacterium in the sample in each vessel will only be lysed if contacted by an enzyme specific to that bacterium. For example, if the sample contains MRSA and the sample is aliquoted into four different vessels, and each vessel is contacted with a different enzyme, the only vessel in which the MRSA will be lysed is the vessel contacted with the MRSA-specific lytic enzyme sources from phage or bacterium. The MRSA in the remaining three vessels will not be lysed because it has been contacted with lysing enzymes that are not specific to MRSA. If the bacterium present in the sample in the vessel is lysed by the enzyme added to that vessel, the intracellular genes or gene products of that bacterium will be exposed.

The sample in each vessel is then contacted by a particle having a protein on a surface of the particle. The particle can be any type of particle that expresses a surface protein, such as Protein A, Protein G, or Protein L, or is capable of be coupled to the protein. Exemplary particles include beads that are capable of being coupled to a protein, such as latex beads, resin beads, magnetic beads, gold beads, polymer beads, or any type of bead known in the art. The bead has a protein, such as Protein A, Protein G, or Protein L, coupled to the surface of the bead. The particle can also be a live or heat killed bacterium that has been engineered with a recombinant plasma to over-express a surface protein, such as Protein A, Protein G, or Protein L. The live bacterium should be an innocuous bacterium, such as Lactococcus or Streptococcus gordonii.

In certain embodiments, the live or heat killed bacterium added to each vessel should be a bacterium that is unaffected by the bacterium-specific lytic enzyme, i.e., is not lysed by the enzyme. Thus the live or heat-kilted bacterium should be different from the enzyme added to that vessel. For example, if the enzyme added to the vessel is a MRSA-specific lysing enzyme, such as ClyS, MV-L (Rashel, J. Infect. Dis. 196:1237-1247, 2005) or lysostaphin, the live or heat-killed bacterium to be contacted to the sample in that vessel should be any bacterium except MRSA, such as Lactococcus, Streptococcus gordonii, Group A Streptococcus, Enterococcus, Pneumococcus, Group B Streptococcus, or Bacillus anthracis.

In other embodiments, the live or heat-killed bacterium can be any bacterium, even a bacterium that is the same as the enzyme added to the vessel. For example, if the enzyme added to the vessel is a GBS-specific lysing enzyme, such as PlyGBS, the live or heat-killed, bacterium to be contacted to the sample can be any bacterium, including GBS. In these embodiments, the sample is contacted with an agent that inactivates the bacterium-specific lytic enzyme, prior to the sample being contacted by the live or heat-killed bacterium. Thus the live or heat-killed bacterium is not effected, i.e., not lysed, by the bacterium-specific lytic enzyme because the enzyme has been inactivated. Inactivation of the bacterium-specific lytic enzyme can be accomplished by any method known in the art, such as adding a buffer to the sample that inactivates the enzyme or adding a protease inhibitor to the sample.

A different antibody is then added to the sample in each vessel. The antibody added to a particular vessel depends on the enzyme that was added to that vessel. The antibody added to a particular vessel should be correlated with the enzyme that was added to that vessel. For example, a vessel that had a MRSA-specific lysing enzyme added to it, should have an antibody specific for the intracellular genes and gene products of MRSA added to it, or a vessel that had a GBS-specific lysing enzyme added to it, should have an antibody specific for the intracellular genes and gene products of GBS added to it.

The vessels are visually observed for presence of agglutination. Agglutination indicates that the antibody carrying particles have cross-linked with the intracellular gene or gene product of the lysed bacterium in that vessel, leading to solid panicles coming out of solution and becoming visible flecks on the slide. Only the vessel containing lysed bacterium will show agglutination. The bacterium is identified by correlating the vessel in which agglutination is observed with the enzyme or antibody added to that vessel.

Another aspect of the invention provides a method of determining presence of methicillin-resistant S. aureus in a sample from a subject and distinguishing methicillin-resistant S. aureus from Staphylococcus epidermidis. The mecA gene that encodes PBP2A in MRSA is also found in a related bacterium, S. epidermidis. However. MRSA is coagulase positive whereas S. epidermidis is not. Therefore, a method including a second agglutination step involving an anti-coagulase antibody would indicate presence of MRSA instead of S. epidermidis. The method involves aliquoting a sample from a subject into a first aliquot and a second aliquot; contacting the first aliquot with S. aureus-specific lytic enzyme to lyse S. aureus in the sample if present, thereby exposing an intracellular gene or gene product of the S. aureus, and detecting the presence of the intracellular gene or gene product by an immunoassay; contacting the second aliquot with an anti-coagulase antibody; and observing the first and second aliquots for presence of agglutination; in which agglutination in both the first and second aliquots indicates presence of MRSA.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance which can be adapted to the practice of this invention in its various embodiments and equivalents thereof.

### EXAMPLES

### Example 1

### Detecting a Bacterium in a Sample from a Subject

A sterile swab is placed and swirled sequentially inside both nasal cavities (Anterior Nares) of a subject for five seconds in each nostril. Other body sites for testing include the axilla (arm pit) and the inguinal area, (groin). The swabbed end is then placed in a test tube containing 200-400 μl of reagent 1 containing a sufficient strength of the MRSA-specific phage lysing enzyme ClyS, MV-L or lysostaphin in the vessel A protease inhibitor is added to the vessel to maintain the integrity of the PBP2a enzyme.

After the swab is immersed in reagent 1 for about one to about three minutes, the swab is swirled for an additional ten to fifteen seconds, and the swab is then removed from the vessel, leaving an aqueous solution of reagent 1.

A drop (approximately 100 μl) of reagent 1 (containing the swab eluant) is added to a left side of a glass slide. A drop of reagent 2 is then added to the drop of reagent 1 on the glass slide. Reagent 2 contains antibodies specific to PBP2A that are attached to a live or heat killed Lactococcus lactis organism, in a suspension of a sufficient number of bacteria per ml of preservatives and sterile water. A buffer may be used instead of sterile water. The control will be sterile water or buffer without airy swab material followed by a drop of reagent 2. The control reaction can be performed on the right side of the glass slide or on a separate slide. A tooth pick is used to swirl the two reagents together.

The drops are visually observed for presence of agglutination. Agglutination indicates that the antibody carrying particles (Lactococcus lactis) have cross-linked with PBP2A, leading to solid particles coming out of solution and becoming visible flecks on the slide. The negative control will remain a homogeneous suspension.

### Example 2

### Identifying a Bacterium in a Sample from a Subject Using Multiple Enzymes and Multiple Antibodies

A sterile swab is placed and swirled sequentially inside both nasal cavities (Anterior Nares) of a subject for five seconds in each nostril. Other body sites for testing include the axilla (arm pit) and the inguinal area (groin). The swabbed end is then placed in a test tube containing 200-400 μl of sterile water or a buffer solution. After the swab is immersed in the tube for about one to about three minutes, the swab is swirled for an additional ten to fifteen seconds, and the swab is then removed from the tube. Half of the volume of the sample is titrated, a second tube.

A different enzyme is then added to each vessel. Reagent 1 contains a sufficient strength of the MRSA-specific lysing enzyme such as ClyS, MV-L or lysostaphin, which is added, to the first vessel (200-400 μl). Reagent 2 contains a sufficient strength of a different bacterium-specific phage lysing enzyme, PlyGBS in this case, which is added to the second vessel (200-400 μl).

A different antibody is then added to each vessel. The antibody to be added to each vessel correlates with the enzyme that is added to that vessel. Reagent 3 contains multiple but distinct monoclonal antibodies specific to PBP2A attached to live or heat killed Lactococcus lactis organisms in a suspension of a sufficient number of bacteria per ml of preservatives and sterile water. Buffer may be used instead of sterile water. Reagent 4 contains multiple but distinct monoclonal antibodies specific to sspB1 attached to live or heat killed Lactococcus lactis organisms in a suspension of a sufficient number of bacteria per ml of preservatives and sterile water. Buffer may be used instead of sterile water. Protease inhibitor is added to each vessel to maintain the integrity of the enzyme added to each vessel.

A drop (approximately 100 μl) of reagent 3 is added to the first vessel. The antibody of reagent 3 (antibodies specific to PBP2A) correlates with the enzyme of reagent 1 (MRSA-specific lysing enzyme ClyS, MV-L or lysostaphin). A drop (approximately 100 μl) of reagent 4 is added to the second vessel. The antibody of reagent 4 (antibodies specific to sspB1) correlates with the enzyme of reagent 2 (bacterium-specific phage lysing enzyme, PlyGBS). There is a control for each vessel. The first control will be sterile water or buffer without any swab material followed by a drop of reagent 3. The second control will be sterile water or buffer without any swab material followed by a drop of reagent 4. A tooth pick is used to swirl each vessel.

The tubes and slides are visually observed for presence of agglutination. Agglutination indicates that the antibody carrying particles (Lactococcus lactis) have cross-linked with the intracellular gene or gene product of that tube, leading to solid particles coming out of solution and becoming visible flecks in the tube. The negative control will remain a homogeneous suspension. Only the tube containing lysed bacterium will show agglutination. The bacterium is identified by correlating the vessel in which agglutination is observed with the enzyme or antibody added to that tube.

### Example 3

### Detecting a Bacterium in a Sample from a Subject

A sterile swab is placed and swirled sequentially inside both nasal cavities (Anterior Nares) of a subject for five seconds in each nostril. Other body sites for testing include the axilla (arm pit) and the inguinal area (groin). The swabbed end is then placed in a test tube containing 200-400 μl of reagent 1 containing a sufficient strength of the MRSA-specific lysing enzyme ClyS, MV-L or lysostaphin in the vessel A protease inhibitor is added to the vessel to maintain the integrity of the PBP2a enzyme.

After the swab is immersed in reagent 1 for about one to about three minutes, the swab is swirled for art additional ten to fifteen seconds, and the swab is then removed from the vessel, leaving an aqueous solution of reagent 1.

A drop (approximately 100 μl) of reagent 1 (containing the swab eluant) is added to a left side of a glass slide. A buffer or an additional protease inhibitor is added to inactivate ClyS or MV-L.

A drop of reagent 2 is then added to the drop of reagent 1 on the glass slide. Reagent 2 contains antibodies specific to PBP2A that are attached to a live or heat killed organism in a suspension of a sufficient number of bacteria per ml of preservatives and sterile water. A buffer may be used instead of sterile water. The control will be sterile water or buffer without any swab material followed by a drop of reagent 2. The control reaction can be performed on a right side of the glass slide or on a separate slide. A tooth pick is used to swirl the two reagents together.

The drops are visually observed for presence of agglutination. Agglutination indicates that the antibody carrying particles have cross-linked with PBP2A, leading to solid particles coming out of solution and becoming visible flecks on the slide. The negative control will remain a homogeneous suspension.

### Example 4

### Identifying a Bacterium in a Sample from a Subject Using Multiple Enzymes and Multiple Antibodies

A sterile swab is placed and swirled sequentially inside both nasal cavities (Anterior Nares) of a subject for five seconds in each nostril. Other body sites for testing include the axilla (arm pit) and the inguinal area (groin). The swabbed end is then placed in a test tube containing 200-400 μl of sterile water or a buffer solution. After the swab is immersed in the tube for about one to about three minutes, the swab is swirled for an additional ten to fifteen seconds, and the swab is then removed from the tube. Half of the volume of the sample is titrated a second tube.

A different enzyme is then added to each tube. Reagent 1 contains a sufficient strength of the MRSA-specific lysing enzyme such as ClyS, MV-L or lysostaphin, which is added to the first tube (200-400 μl). Reagent 2 contains a sufficient strength of a different bacterium-specific phage lysing enzyme, PlyGBS in this case, which is added to the second tube (200-400 μl). A buffer or an additional protease inhibitor is added to each tube to inactivate the enzymes.

A different antibody is then added to each tube. The antibody to be added to each tube correlates with the enzyme that is added to that tube. Reagent 3 contains multiple but distinct monoclonal antibodies specific to PBP2A attached to live or heat killed organisms in a suspension of a sufficient number of bacteria per ml of preservatives and sterile water. Buffer may be used instead of sterile water. Reagent 4 contains multiple but distinct monoclonal antibodies specific to sspB1 attached to live or heat killed organisms in a suspension of a sufficient number of bacteria per ml of preservatives and sterile water. Buffer may be used instead of sterile water.

A drop (approximately 100 μl) of reagent 3 is added to the first, tube. The antibody of reagent 3 (antibodies specific to PBP2A) correlates with the enzyme of reagent 1 (MRSA-specific phage lysing enzyme such as ClyS, MV-L or lysostaphin). A drop (approximately 100 μl) of reagent 4 is added to the second tube. The antibody of reagent 4 (antibodies specific to sspB1) correlates with the enzyme of reagent 2 (bacterium-specific lysing enzyme, PlyGBS). There is a control for each tube. The first, control will be sterile water or buffer without any swab material followed by a drop of each of reagent 3. The second control will be sterile water or buffer without any swab material followed by a drop of each of reagent 4. A tooth pick is used to swirl each tube.

The tubes are visually observed for presence of agglutination. Agglutination indicates that the antibody carrying particles have cross-linked with the intracellular gene or gene product of that tube, leading to solid particles coming out of solution and becoming visible flecks on the slide. The negative control will remain a homogeneous suspension. Only the tube containing lysed bacterium will show agglutination. The bacterium is identified by correlating the tube in which agglutination is observed with the enzyme or antibody added to that tube.

### Example 5

### Expression of Localization of Protein A in L. lactis

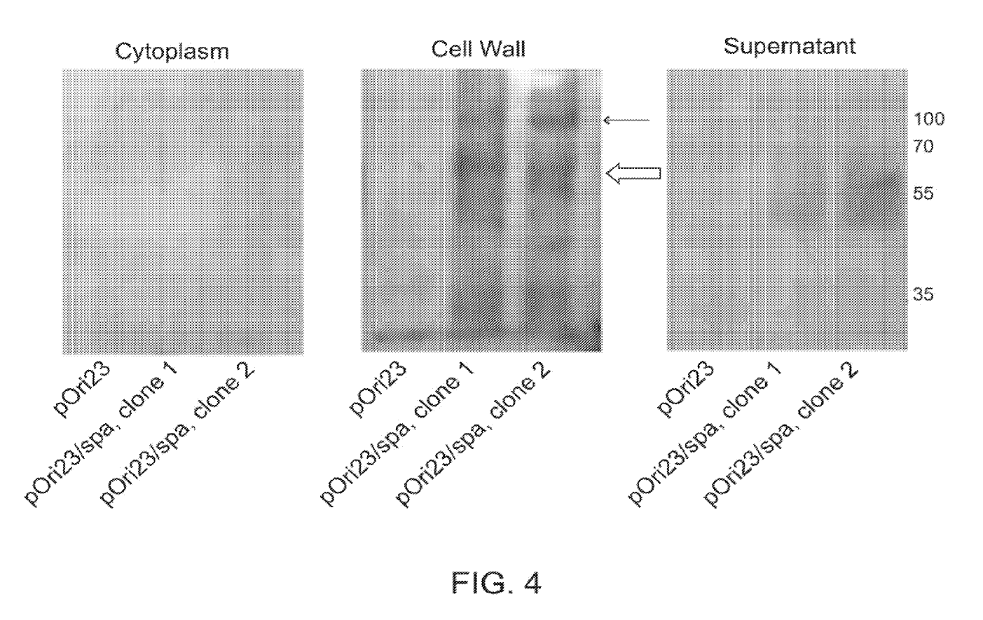

The protein A gene (spa) from MRSA252 (a larger spa gene with five IgG binding domains) has been cloned into shuttle plasmid pOri23 carrying a moderate strength lactococcal promoter in E. coli (Que, Infect. Immun. 68:35616-3522, 2000). To optimize surface expression, the ribosomal binding site and signal sequence of spa was replaced with one from L. lactis. L. lactis strains MG1363 was subsequently transformed with the recombinant pOri23 carrying the spa gene (Wells, Appl. Environ. Microbiol. 59:3954-3959, 1993). Expression and localization studies confirmed that Spa is displayed on the lactococcal surface (FIG. 4).

### Example 6

### The Binding of a Fixed Number of Protein A-Expressing L. lactis to FITC-Conjugated IgG from Different Mammalian Species

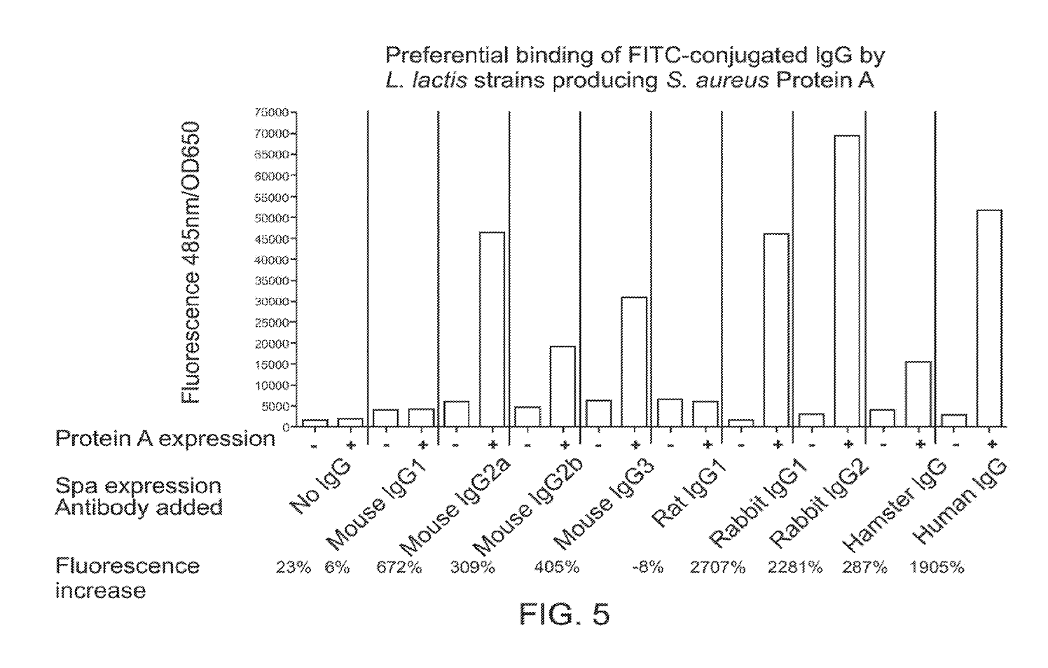

As Spa (protein A) binds to diverse species of IgGs with varying affinities (40a), an assay to determine the binding of Spa anchored on the surface of L. lactis to various IgGs was developed, especially from those mammalian species in which monoclonal antibodies are to be raised (i.e. mouse, rabbit and human monoclonals). Using a fixed number of L. lactis cells and FITC-labeled IgG, it was found that rabbit IgG1, IgG2 and whole human IgG bound Spa on L. lactis much better than murine IgG2a, 2b and 3 (FIG. 5). In addition, rabbit IgG exhibited better binding to immobilized Spa than human IgG. While these studies imply that MAbs (IgG1 and 2) from rabbit likely bind better to Spa-expressing L. lactis cells than human IgG (FIG. 5). Together, these data suggested it is better to produce rabbit or human monoclonal antibodies to PBP2a in the detection of MRSA.

### Example 7

### Purification of PBP2a from E. coli

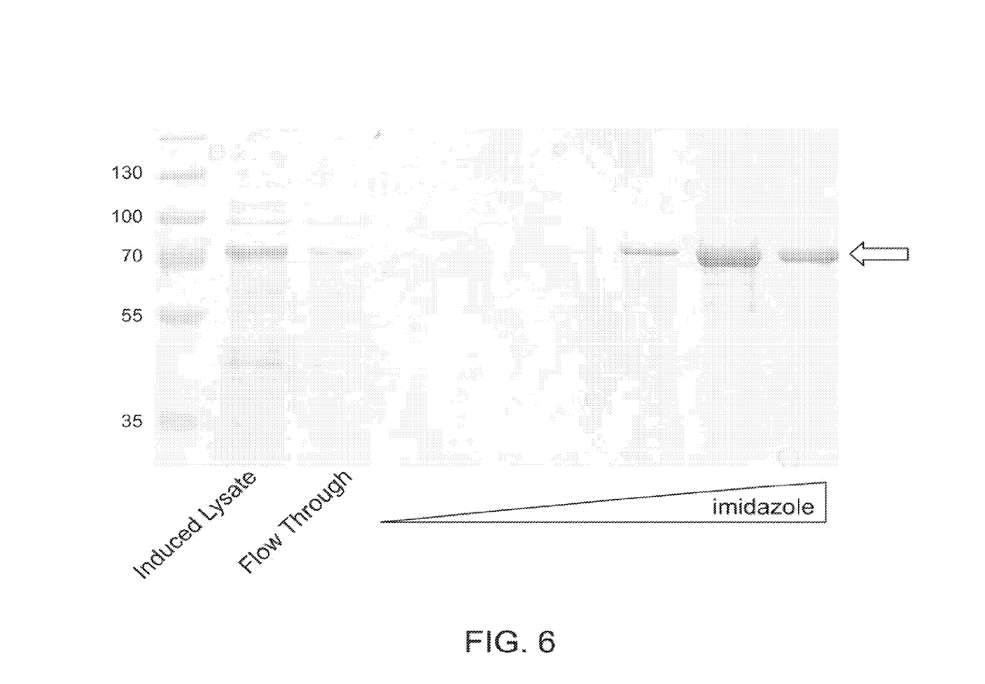

The cytoplasmic portion of the mecA encoding PBP2a (residues 23-668 where residues 1-22 is the transmembrane domain) has been cloned, into expression vector pET14b in E. coli, expressed under IPTG-inducing condition and purified over a nickel column, following previously described protocol for purification of PBP2a (Lim, Nat. Struct. Biol. 11:870-876, 2002). Analysis of fractions in an SDS-gel confirmed the purity of the protein (FIG. 6). The authenticity of the protein was verified by MS/MS analysis. PBP2a obtained in this manner can be used for immunization to yield antibodies from appropriate, animal species.

### Example 8

### Agglutination Reaction of the OVA Antigen with Rabbit Anti-OVA Antibodies Attached to Protein A-Expressing L. lactis

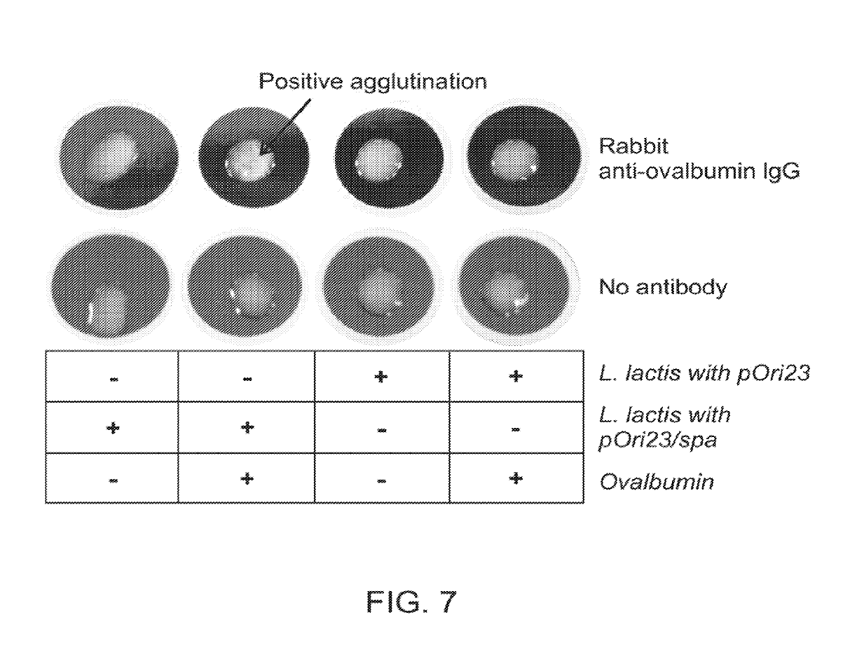

To test the feasibility of the agglutination reaction using L. lactis, OVA antigen was used, which is a well characterized antigen in immunological assays and to which specific antibodies are plentifully available. Using polyclonal rabbit anti-OVA antibody attached to L. lactis expressing protein A on it surface as the agglutination agent in an about 100 μl volume on a test slide, purified OVA antigen was added to the drop of the agglutination reagent, agglutination was observed to place while the control without the anti-OVA antibodies did not show agglutination (FIG. 7).

What is claimed is:

1. A method of detecting presence of a bacterium in a sample from a subject, the method comprising:
contacting a sample from a subject with a bacterium-specific lytic enzyme capable of specific lysis of a first bacterium if present in the sample, thereby exposing an intracellular gene or gene product of the first bacterium;
contacting the sample with a particle having a protein on a surface of the particle in a presence of an antibody in which an Fc portion specifically binds the protein and an F(ab)₂ portion specifically binds the intracellular gene or gene product of the first bacterium, with the proviso that when the particle is a second bacterium, the second bacterium is different from the first bacterium; and
detecting the presence or absence of the first bacterium by observing the sample for an agglutination reaction, wherein agglutination indicates the presence of the first bacterium in the sample.

2. The method of claim 1, wherein prior to contacting the sample with the enzyme, the method further comprises obtaining the sample from the subject.

3. The method of claim 2, wherein the bacterium-specific lytic enzyme is obtained from a phage.

4. The method of claim 1, wherein the particle is a bead or a second bacterium that over-expresses the protein.

5. The method of claim 4, wherein the second bacterium is heat-killed bacterium that over-expresses the protein or a live bacterium that over-expresses the protein.

6. The method of claim 5, wherein the live bacterium is an innocuous bacterium.

7. The method of claim 6, wherein the innocuous bacterium is Lactococcus or Streptococcus gordonii.

8. The method of claim 1, wherein the sample is a human tissue or body fluid.

9. The method of claim 1, wherein the antibody is murine antibody.

10. The method of claim 9, wherein the antibody is a humanized murine antibody

11. The method of claim 1, wherein the antibody is rabbit antibody.

12. The method of claim 1, wherein the antibody is human antibody.

13. The method of claim 1, wherein the antibody is a monoclonal antibody or a collection of monoclonal antibodies specific for different epitopes of the same intracellular gene product.

14. The method of claim 1, wherein the antibody is a highly specific polyclonal antibody.

15. The method of claim 1, wherein the protein is Protein A or Protein G.

16. The method of claim 1, wherein the bacterium is selected from the group consisting of: methicillin-resistant S. aureus, Group A Streptococcus, vancomycin resistant Enterococcus, Pneumococcus, Group B Streptococcus, Bacillus anthracis, and drug resistant tuberculosis.

17. The method of claim 16, wherein the bacterium is methicillin-resistant S. aureus.

18. The method of claim 17, wherein the bacterium-specific lytic enzyme is S. aureus-specific phage lysin or lysostaphin.

19. The method of claim 18, wherein the particle is a heat-killed bacterium that over-expresses Protein A or Protein G.

20. The method of claim 18, wherein the antibody is specific for a protein coming from a SCCmec cassette.

21. The method of claim 20, wherein the protein coming from the SCCmec cassette is PBP2A.

22. The method of claim 20, wherein agglutination indicates the presence of methicillin-resistant S. aureus in the sample.

23. A method of identifying a bacterium in a sample from a subject, the method comprising:
aliquoting a sample into at least two vessels;
contacting the sample in each vessel with a different bacterium-specific lytic enzyme, thereby exposing an intracellular gene or gene product of a first bacterium in the vessel if the first bacterium is lysed by the particular enzyme added to that vessel;
contacting the sample in each vessel with a particle having a protein on a surface of the particle, with the proviso that when the particle is a second bacterium, the second bacterium is not lysed by the enzyme that was added to that vessel;
contacting the sample in each vessel with a different antibody, wherein the antibody added to each vessel is correlated with the enzyme that was added to that vessel;
observing each vessel for presence of an agglutination reaction, wherein agglutination indicates presence of the first bacterium in that vessel; and
identifying the first bacterium by correlating the vessel in which agglutination was observed with the enzyme or antibody added to that vessel.

24-39. (canceled)

40. A method of detecting presence of a bacterium in a sample from a subject, the method comprising:
contacting a sample from a subject with a bacterium-specific lytic enzyme capable of specific lysis of a first bacterium if present in the sample, thereby exposing an intracellular gene or gene product of the first bacterium;
inactivating the enzyme;
contacting the sample with a second bacterium that over-expresses a surface protein in a presence of an antibody in which an Fc portion specifically binds the protein and an F(ab)₂ portion specifically binds the intracellular gene or gene product of the first bacterium;
detecting the presence or absence of the first bacterium by observing the sample for an agglutination reaction, wherein agglutination indicates the presence of the bacterium in the sample.

41-57. (canceled)

58. A method of identifying a bacterium in a sample from a subject, the method comprising:
aliquoting a sample into at least two vessels;
contacting the sample in each vessel with a different bacterium-specific lytic enzyme, thereby exposing an intracellular gene or gene product of a first bacterium in the vessel if the first bacterium is lysed by the particular enzyme that was added to that vessel;
inactivating the enzyme in each vessel;
contacting the sample in each vessel with a second bacterium that over-expresses a surface protein;
contacting the sample in each vessel with a different antibody, wherein the antibody added to each vessel is correlated with the enzyme that was added to that vessel;
observing each vessel for presence of an agglutination reaction, wherein agglutination indicates presence of the first bacterium in that vessel; and
identifying the first bacterium by correlating the vessel in which agglutination was observed with the enzyme or antibody added to that vessel.

59-99. (canceled)

100. A kit for detecting methicillin-resistant S. aureus, the kit comprising:
S. aureus-specific phage lysin or lysostaphin;
at least one particle having a protein on a surface of the particle; and
at least one antibody in which a Fc portion specifically binds the protein and a F(ab)₂ portion specifically binds an intracellular gene or gene product of S. aureus.

101-114. (canceled)

115. A kit for detecting a bacterium, the kit comprising:
at least one bacterium-specific lytic enzyme;
at least one particle having a protein on a surface of the particle; and
at least one antibody in which a Fc portion specifically binds the protein and a F(ab)₂ portion specifically binds an intracellular gene or gene product of a bacterium lysed by the enzyme.

116-132. (canceled)
